(19) United States
(12) Patent Application Publication
(10) Pub. No.: US 2026/0306010 A1
(43) Pub. Date: Oct. 8, 2026

(54) PARKINSON'S TREMOR CLASSIFICATION USING MACHINE LEARNING

(71) Applicant: Genzyme Corporation, Cambridge, MA (US)

(72) Inventors: Haneen Njoum, Cambridge, MA (US); Hillol Sarker, Cambridge, MA (US); Elias Abou Zeid, Cambridge, MA (US)

(21) Appl. No.: 19/481,988

(22) PCT Filed: May 3, 2024
(86) PCT No.: PCT/US2024/027768

§ 371 Date: Nov. 6, 2025

Related U.S. Application Data
(60) Provisional application No. 63/502,033, filed on May 12, 2023.

(51) Int. Cl. A61B 5/00; A61B 5/11; A61B 5/397; G06T 7/00; G06T 7/20

(52) U.S. Cl. CPC A61B 5/4082; A61B 5/0077; A61B 5/1101; A61B 5/1128; A61B 5/397; A61B 5/7267; G06T 7/0012; G06T 7/20; A61B 2562/0219; A61B 2576/00; G06T 2207/10016; G06T 2207/20084; G06T 2207/30004

(57) ABSTRACT

Methods, systems, and apparatus, including computer programs encoded on a computer storage medium, for classifying a tremor of a subject having Parkinson's disease. In one aspect, there is provided a method comprising: receiving accelerometer data characterizing motion of the subject having Parkinson's disease; processing the accelerometer data using a tremor classification neural network, in accordance with values of a set of tremor classification neural network parameters, to generate a score distribution over a set of tremor classes, wherein the set of tremor classes comprises multiple tremor classes, wherein each tremor class in the set of tremor classes corresponds to a respective severity of tremor; and classifying a tremor of the subject having Parkinson's disease based on the score distribution over the set of tremor classes.

### CROSS REFERENCE TO RELATED APPLICATIONS

The present application is the national stage entry of International Patent Application No. PCT/US2024/027768, filed on May 3, 2024, and claims priority to Application No. 63/502,033, filed on May 12, 2023, the disclosures of which are incorporated herein by reference.

### TECHNICAL FIELD

This specification relates to Parkinson's tremor classification using machine learning.

### BACKGROUND

Machine learning models receive an input and generate an output, e.g., a predicted output, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

Parkinson's disease is a chronic, progressive neurological disorder caused by the gradual loss of nerve cells in the brain. The loss of nerve cells causes a deficit of dopamine, a neurotransmitter that is responsible for controlling movement. When nerve cells degenerate or die, the body has difficulty controlling movement and the symptoms of Parkinson's begin to appear. Common symptoms of Parkinson's disease include tremors, slowness and stiffness of movement, difficulty with balance and coordination, and an inability to make facial expressions. In advanced cases, patients may also suffer from cognitive decline, including depression and dementia. Diagnosis of Parkinson's is made through a neurological exam, medical history, and imaging tests.

Dyskinesia is a movement disorder characterized by involuntary and often irregular movements, such as writhing, jerking, twisting, or wriggling. It is a common side effect of long-term use of medications that affect dopamine levels in the brain, such as those used to treat Parkinson's disease.

### SUMMARY

This specification describes a classification system implemented as computer programs on one or more computers in one or more locations that can process accelerometer data characterizing motion of a subject with Parkinson's disease to classify a tremor of the subject, or to classify dyskinesia of the subject, or both.

Throughout this specification, a “subject” refers to a human subject, i.e., a person.

Throughout this specification, a “subnetwork” of a neural network refers to a portion of the neural network.

Throughout this specification, an “embedding” of an entity (e.g., of an accelerometer signal) refers to a representation of the entity in a latent space that is generated by a neural network (or a subnetwork of a neural network). An embedding can be represented as an ordered collection of numerical values, e.g., a vector, matrix, or other tensor of numerical values.

Throughout this specification, a “block” (e.g., a “convolutional block” or a “recurrent block”) refers to a group of neural network layers in a neural network.

Throughout this specification, the terms “accelerometer data” and “accelerometer signal” are used interchangeably.

In one aspect, there is provided a method performed by one or more computers, the method comprising: receiving accelerometer data characterizing motion of a subject having Parkinson's disease; processing the accelerometer data using a tremor classification neural network, in accordance with values of a set of tremor classification neural network parameters, to generate a score distribution over a set of tremor classes, wherein the set of tremor classes comprises multiple tremor classes, wherein each tremor class in the set of tremor classes corresponds to a respective severity of tremor; and classifying a tremor of the subject having Parkinson's disease based on the score distribution over the set of tremor classes.

In some implementations, the accelerometer data is generated by a wearable device of the subject.

In some implementations, the accelerometer data is represented as one or more one-dimensional (1D) temporal signals.

In some implementations, the accelerometer data is represented as a two-dimensional (2D) spectrogram signal.

In some implementations, the set of tremor classes comprises at least three tremor classes.

In some implementations, the set of tremor classes corresponds to a clinical tremor scale.

In some implementations, each tremor class in the set of tremor classes corresponds to a respective magnitude, frequency, and duration of tremor.

In some implementations, the method further comprises receiving video data characterizing motion of the subject; wherein processing the accelerometer data using the tremor classification neural network comprises: jointly processing the accelerometer data and the video data using the tremor classification neural network.

In some implementations, the method further comprises receiving surface electromyography (EMG) data characterizing electrical potentials produced during muscle contractions in the subject; wherein processing the accelerometer data using the tremor classification neural network comprises: jointly processing the accelerometer data and the surface EMG data using the tremor classification neural network.

In some implementations, processing the accelerometer data using the tremor classification neural network to generate the score distribution over the set of tremor classes comprises: processing the accelerometer data using a convolutional block, comprising one or more convolutional neural network layers, to generate a convolutional block output; processing the convolutional block output using a recurrent block, comprising one or more recurrent neural network layers, to generate a recurrent block output; and processing the recurrent block output using a dense block, comprising one or more dense neural network layers, to generate the score distribution over the set of tremor classes.

In some implementations, the convolutional block output has a lower temporal resolution than the accelerometer data.

In some implementations, the recurrent neural network layers are long short-term memory (LSTM) neural network layers.

In some implementations, classifying the tremor of the subject having Parkinson's disease based on the score distribution over the set of tremor classes comprises: classifying the tremor of the subject into a tremor class associated with a highest score from among the set of tremor classes.

In some implementations, the method further comprises: repeatedly performing the tremor classification over a sequence of time intervals to generate a sequence of tremor classifications, wherein each tremor classification in the sequence of tremor classifications corresponds to a respective time interval and classifies a tremor of the subject during the time interval.

In some implementations, the method further comprises: processing the sequence of tremor classifications to classify a progression of Parkinson's disease in the subject into a progression state from a set of progression states.

In some implementations, the method further comprises: administering a drug for treatment of Parkinson's disease or symptoms of Parkinson's disease to the subject based at least in part on the progression state of Parkinson's disease in the subject.

In some implementations, the method further comprises: generating a notification that indicates the classification of the tremor in the subject having Parkinson's disease.

In some implementations, processing the accelerometer data using the tremor classification neural network to generate the score distribution over the set of tremor classes comprises: processing the accelerometer data using an encoder subnetwork of the tremor classification neural network to generate an embedding of the accelerometer data in a latent space; and processing the embedding of the accelerometer data in the latent space using a tremor subnetwork of the tremor classification neural network to generate the score distribution over the set of tremor classes.

In some implementations, the method further comprises: processing the embedding of the accelerometer data in the latent space using a dyskinesia subnetwork of the tremor classification neural network to generate a score distribution over a set of dyskinesia classes, wherein the set of dyskinesia classes comprises multiple dyskinesia classes, wherein each dyskinesia class in the set of dyskinesia classes corresponds to a respective severity of dyskinesia.

In some implementations, the method further comprises: classifying dyskinesia of the subject having Parkinson's disease based on the score distribution over the set of dyskinesia classes.

In some implementations, the tremor classification neural network has been trained by operations comprising: pre-training the encoder subnetwork of the tremor classification neural network to perform an auxiliary task, wherein the auxiliary task is not a tremor classification task; and after pre-training the encoder subnetwork, training the tremor classification neural network to perform the tremor classification task.

In some implementations, pre-training the encoder subnetwork of the tremor classification neural network to perform the auxiliary task comprises: obtaining a base accelerometer signal; generating a positive pair of embeddings in the latent space, comprising: processing a first transformed version of the base accelerometer signal using the encoder subnetwork to generate a corresponding embedding in the latent space; processing a second transformed version of the base accelerometer signal using the encoder subnetwork to generate a corresponding embedding in the latent space; training the encoder subnetwork to optimize an auxiliary loss that depends on: (i) the embedding of the first transformed version of the base accelerometer signal, and (ii) the embedding of the second transformed version of the base accelerometer signal.

In some implementations, the method further comprises generating the first transformed version of the base accelerometer signal, comprising: randomly sampling a first transformation from a space of transformations; and applying the first transformation to the base accelerometer signal to generate the first transformed version of the base accelerometer signal.

In some implementations, the method further comprises generating the second transformed version of the base accelerometer signal, comprising: randomly sampling a second transformation from a space of transformations; and applying the second transformation to the base accelerometer signal to generate the second transformed version of the base accelerometer signal.

In some implementations, the auxiliary loss measures an error between: (i) embedding of the first transformed version of the base accelerometer signal, and (ii) the embedding of the second transformed version of the base accelerometer signal.

In some implementations, pre-training the encoder subnetwork of the tremor classification neural network to perform the auxiliary task comprises: obtaining an accelerometer signal; generating a masked accelerometer signal by masking a portion of the accelerometer signal; processing the masked accelerometer signal using the encoder subnetwork to generate an embedding of the masked accelerometer signal; processing the embedding of the masked accelerometer signal using a decoder neural network to generate a predicted reconstruction of the accelerometer signal; and jointly training the encoder subnetwork and the decoder neural network to optimize an auxiliary loss that measures an error in the predicted reconstruction of the accelerometer signal.

In some implementations, pre-training the encoder subnetwork of the tremor classification neural network to perform the auxiliary task comprises: obtaining an accelerometer signal; generating a noised accelerometer signal by adding noise the accelerometer signal; processing the noised accelerometer signal using the encoder subnetwork to generate an embedding of the noised accelerometer signal; processing the embedding of the noised accelerometer signal using a decoder neural network to generate a de-noised accelerometer signal; jointly training the encoder subnetwork and the decoder neural network to optimize an auxiliary loss that measures an error in the de-noised accelerometer signal.

In some implementations, pre-training the encoder subnetwork of the tremor classification neural network to perform the auxiliary task comprises: obtaining: (i) an accelerometer signal, and (ii) a target label for the accelerometer signal; processing the accelerometer signal using the encoder subnetwork to generate an embedding of the accelerometer signal; processing the embedding of the accelerometer signal using a prediction neural network to generate a predicted label for the accelerometer signal; and jointly training the encoder subnetwork and the prediction neural network to optimize an auxiliary loss that measures an error between: (i) the target label, and (ii) the predicted label.

In some implementations, the target label for the accelerometer signal defines a number of steps taken by the subject in a duration of time covered by the accelerometer signal.

In some implementations, the target label for the accelerometer signal defines an action performed by the subject in a duration of time covered by the accelerometer signal.

In some implementations, training the tremor classification neural network to perform the tremor classification task comprises: obtaining: (i) a training accelerometer signal, and (ii) a target tremor classification for the training accelerometer signal; processing the training accelerometer signal using the embedding subnetwork to generate an embedding of the training accelerometer signal; processing the embedding of the training accelerometer signal using the tremor subnetwork to generate a score distribution over the set of tremor classes; and training the tremor subnetwork of the tremor classification neural network to optimize a tremor objective function that measures an error between: (i) the score distribution over the set of tremor classes, and (ii) the target tremor classification.

In some implementations, parameter values of the encoder subnetwork are frozen during the training of the tremor classification neural network to perform the tremor classification task.

According to another aspect, there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect, there are provided one or more non-transitory computer storage media storing instructions that when executed by one or more computers cause the one or more computers to perform operations of the methods described herein.

Particular embodiments of the subject matter described in this specification can be implemented so as to realize one or more of the following advantages.

The classification system described in this specification can process accelerometer data characterizing motion of a subject using a classification neural network to classify a tremor of the subject, or to classify dyskinesia of the subject, or both. The classification neural network can directly process raw accelerometer data to generate tremor classifications or dyskinesia classifications without the need for manual or heuristic feature engineering. More specifically, the classification neural network can be trained, by machine learning training techniques, to identify and extract relevant feature representations from raw accelerometer data in order to accurately classify tremors and dyskinesia.

The classification system enables granular and real-time monitoring of the state and progression of Parkinson's disease in a subject without requiring the subject to regularly see a physician or access a healthcare facility. More specifically, the classification system can automatically process accelerometer data generated by a personal device of a subject, e.g., a wearable device or a smartphone, to generate accurate classifications of tremor and dyskinesia without requiring intermediate analysis by a physician. Further, the classification system enables objective and reproducible assessments of tremor and dyskinesia in subjects, and thus provides an advantage over self-reported assessments (which may be inaccurate and variable across subjects) and assessments performed by physicians (which can be performed only at discrete moments in time, in contrast to the classification system, which provides continuous monitoring).

The classification system can train the classification neural network by a two stage process that involves first pre-training an encoder subnetwork of the classification neural network on one or more auxiliary tasks, and then fine-tuning the classification neural network to perform tremor classification or dyskinesia classification. Pre-training the encoder subnetwork can encourage the encoder subnetwork to generate rich and informative features characterizing the structure of accelerometer data, and can reduce the amount of training data and number of training iterations required for fine-tuning the classification neural network. Pre-training the encoder subnetwork can thus reduce consumption of computational resources, e.g., memory and computing power, during fine-tuning of the classification neural network.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example classification system that includes a classification neural network.

FIG. 2 shows an example architecture of the classification neural network.

FIG. 3 shows an example architecture of an encoder subnetwork included in the classification neural network.

FIG. 4 is a flow diagram of an example process for classifying a tremor of a subject having Parkinson's disease or for classifying dyskinesia of a subject having Parkinson's disease.

FIG. 5 shows an example training system.

FIG. 6 is a flow diagram of an example process for pre-training the encoder subnetwork of the classification neural network to perform a contrastive embedding task.

FIG. 7 is a flow diagram of an example process for pre-training the encoder subnetwork of the classification neural network to perform a masked reconstruction task.

FIG. 8 is a flow diagram of an example process for pre-training the encoder subnetwork of the classification neural network to perform a noisy reconstruction task.

FIG. 9 is a flow diagram of an example process for pre-training the encoder subnetwork of the classification neural network to perform a supervised auxiliary task.

FIG. 10 is a flow diagram of an example process for training the classification neural network to perform a tremor classification task.

FIG. 11 is a flow diagram of an example process for training the classification neural network to perform a dyskinesia classification task.

FIG. 12 illustrates an example of experimental results of the classification system in classifying tremors in subjects with Parkinson's disease.

FIG. 13 illustrates: (i) a wearable device, e.g., worn by a subject with Parkinson's disease; (ii) accelerometer data, generated by an accelerometer in the wearable device, represented as one or more one-dimensional (1D) temporal signals; and (iii) accelerometer data, generated by an accelerometer in the wearable device, represented as a two-dimensional (2D) time-frequency spectrogram.

Like reference numbers and designations in the various drawings indicate like elements.

### DETAILED DESCRIPTION

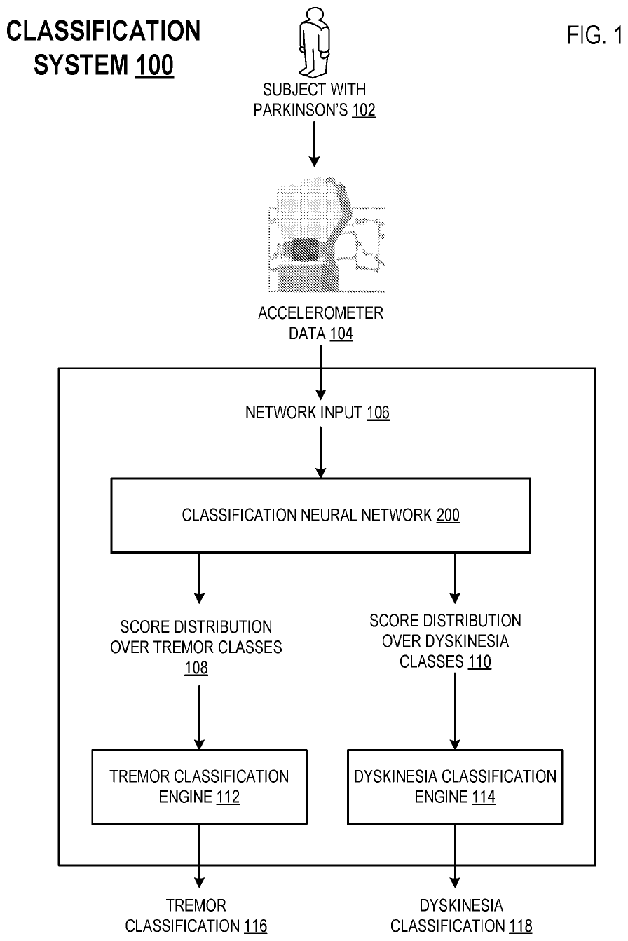

FIG. 1 shows an example classification system 100. The classification system 100 is an example of a system implemented as computer programs on one or more computers in one or more locations in which the systems, components, and techniques described below are implemented.

The classification system 100 is configured to receive accelerometer data 104 characterizing movement of a subject 102 with Parkinson's disease. The classification system 100 processes the accelerometer data 104 to generate one or both of: (i) a tremor classification 116 that characterizes a severity of a tremor of the subject 102, or (ii) a dyskinesia classification 118 that characterizes a severity of (and/or a presence of) dyskinesia in the subject 102.

The accelerometer data 104 can characterize the movement of the subject 102 over an interval of time, e.g., a 1 second, 2 second, 10 second, or 1 minute interval of time. The accelerometer data 104 can be generated by an accelerometer device located on or in proximity to the subject 102. For instance, the accelerometer data 104 can be generated by an accelerometer in a wearable device being worn by the subject 102, e.g., a wearable device worn on the wrist of the subject. As another example, the accelerometer data 104 can be generated by an accelerometer in a portable device (e.g., a smartphone) being held by the subject.

The classification system 100 can represent the accelerometer data 104 in any of a variety of formats. For instance, the classification system 100 can represent the accelerometer data 104 by one or more one-dimensional (1D) temporal signals, e.g., by respective 1D temporal signals that characterizes acceleration in each of the x-direction, the y-direction, and the z-direction (where the x-, y-, and z-directions are defined with reference to an appropriate frame of reference). As another example, the classification system 100 can represent the accelerometer data 104 by a two-dimensional (2D) spectrogram in the time-frequency domain, i.e., that includes one dimension representing time and a second dimension representing frequency.

Optionally, the classification system 100 can receive additional inputs, i.e., in addition to the accelerometer data 104. The additional inputs can include any appropriate data characterizing the subject 102. The classification system 100 can process the additional inputs as part of generating the tremor classification 116, the dyskinesia classification 118, or both. The additional inputs can be derived from any appropriate modalities and can provide the classification system 100 with additional sources of information for increasing the accuracy of the tremor classification 116 and the dyskinesia classification 118. A few examples of additional inputs to the classification system 100 are described next.

In some implementations, the classification system 100 additionally receives video data characterizing the motion of the subject 102. More specifically, the video data can include a video that shows part of the subject (e.g., the hand of the subject, or the face of the subject, or the torso of the subject) or all of the subject (e.g., the entire body of the subject) over an interval of time. The interval of time can be, e.g., a 1 second, 2 second, 10 second, or 1 minute interval of time, and can characterize the subject over the same interval of time as the accelerometer data 104. The video data can be captured by a video recording device, e.g., a webcam, or a video recording device in a smartphone. The video data can be represented as a sequence of video frames captured at any appropriate sampling frequency, e.g., 24 frames-per-second.

In some implementations, the classification system 100 additionally receives surface electromyography (EMG) data characterizing electrical potentials produced during muscle contractions in the subject 102. The surface EMG data can be measured over any appropriate interval of time, e.g., a 1 second, 2 second, 10 second, or 1 minute interval of time, and can characterize the subject over the same time interval as the accelerometer data. Surface EMG data measures electrical activity of the muscles of the subject. Capturing surface EMG data can involve placing electrodes on the surface of the skin over the muscles of interest. These electrodes pick up the electrical activity that is generated when the muscles contract. The classification system 100 can represent the surface EMG data, e.g., by an array of 1D temporal signals, or in any other appropriate format.

The classification system 100 can process the accelerometer data 104, and any additional inputs characterizing the subject 102, using a classification neural network 200, a tremor classification engine 112, and a dyskinesia classification engine 118, which are each described next.

The classification neural network 200 is configured to receive a network input 106 that includes the accelerometer data 104, and optionally, any additional inputs characterizing the subject 102, e.g., video data, surface EMG data, etc. The classification neural network 200 processes the network input 106, in accordance with values of a set of classification neural network parameters, to generate one or both of: (i) a score distribution 108 over a set of tremor classes, and (ii) a score distribution 110 over a set of dyskinesia classes.

The score distribution 108 over the set of tremor classes can include a respective score for each tremor class in the set of tremor classes. The score for a tremor class can define a likelihood that the subject has a tremor that is included in the tremor class. Each tremor class can correspond to a respective severity of tremor. The severity of a tremor can characterize, e.g., the magnitude, frequency, and duration (consistency) of the tremor. The set of tremor classes can include any appropriate number of tremor classes, e.g., three tremor classes, four tremor classes, or five tremor classes. The set of tremor classes can include a “no tremor” class, i.e., indicating that the subject is not exhibiting a tremor. The set of tremor classes can correspond to a clinical tremor scale, e.g., The Essential Tremor Rating Assessment Scale (TETRAS).

The score distribution 110 over the set of dyskinesia classes can include a respective score for each dyskinesia class in the set of dyskinesia classes. The score for a dyskinesia class can define a likelihood that the subject has dyskinesia that is included in the dyskinesia class. Each dyskinesia class can correspond to a respective severity of dyskinesia. The severity of dyskinesia can characterize, e.g., the magnitude, frequency, and duration (consistency) of involuntary writhing, jerking, twisting, or wriggling movements by the subject. The set of dyskinesia classes can include any appropriate number of dyskinesia classes, e.g., three dyskinesia classes, four dyskinesia classes, or five dyskinesia classes. The set of dyskinesia classes can include a “no dyskinesia” class, i.e., indicating that the subject is not exhibiting symptoms of dyskinesia.

In some implementations, the classification neural network 200 generates only a score distribution over the set of tremor classes, and does not generate a score distribution over the set of dyskinesia classes. In some implementations, the classification neural network 200 generates only a score distribution over the set of dyskinesia classes, and does not generate a score distribution over the set of tremor classes. In some implementations, the classification neural network generates both a score distribution over the set of tremor classes and a score distribution over the set of dyskinesia classes.

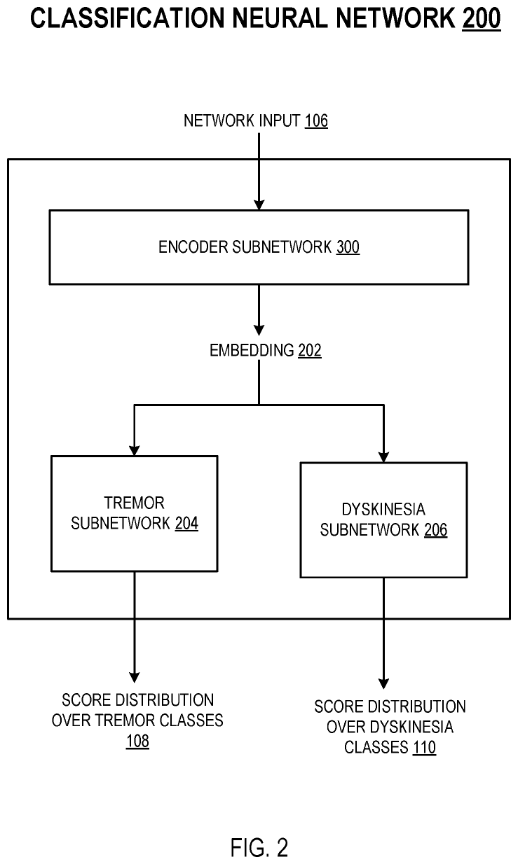

The classification neural network 200 can have any appropriate neural network architecture that enables the classification neural network 200 to perform its described functions, e.g., generating a score distribution over a set of tremor classes, or generating a score distribution over a set of dyskinesia classes, or both. In particular, the classification neural network can include any appropriate neural network layers (e.g., convolutional layers, attention layers, fully connected layers, recurrent layers, etc.) in any appropriate number (e.g., 5 layers, 10 layers, or 50 layers) and connected in any appropriate configuration (e.g., as a linear sequence of layers). An example architecture of the classification neural network is described in more detail with reference to FIG. 2.

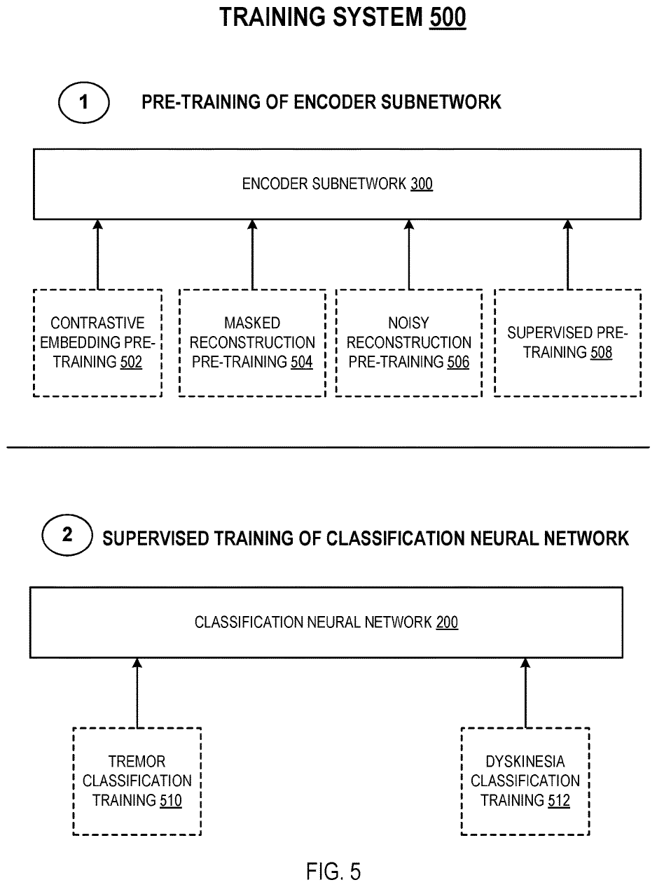

The classification system 100 can use a training system to train the classification neural network 200 to perform the task of tremor classification, or the task of dyskinesia classification, or both. Optionally, the training system can pre-train portions of the classification neural network 200 to perform one or more unsupervised or supervised auxiliary tasks prior to training the classification neural network 200 to perform the tremor classification task, or the dyskinesia classification task, or both. An example of a training system for training the classification neural network is described in more detail with reference to FIG. 5.

The tremor classification engine 112 is configured to process a score distribution 108 over the set of tremor classes to generate a tremor classification 116. The tremor classification engine 112 can, for instance, generate a tremor classification that classifies the subject as being included in the tremor class associated with the highest score under the score distribution 108 over the set of tremor classes.

Optionally, the tremor classification engine 112 can generate a confidence score that characterizes a confidence of the classification system 100 in the tremor classification 116 generated for the subject 102. The tremor classification engine 112 can generate a confidence score for a tremor classification 116 in any appropriate way. For instance, the tremor classification engine 112 can generate a confidence score by computing an entropy of the score distribution over the set of tremor classes 108. In this example, a higher entropy of the score distribution over the set of tremor classes 108 can reflect a higher uncertainty of the classification system 100 in the tremor classification.

The dyskinesia classification engine 118 is configured to process a score distribution 110 over the set of dyskinesia classes to generate a dyskinesia classification 118. The dyskinesia classification engine 118 can, for instance, generate a dyskinesia classification that classifies the subject as being included in the dyskinesia class associated with the highest score under the score distribution 110 over the set of dyskinesia classes.

Optionally, the dyskinesia classification engine 118 can generate a confidence score that characterizes a confidence of the classification system 100 in the dyskinesia classification 118 generated for the subject 102. The dyskinesia classification engine 118 can generate a confidence score for a dyskinesia classification 118 in any appropriate way. For instance, the dyskinesia classification engine 118 can generate a confidence score by computing an entropy of the score distribution over the set of dyskinesia classes 110. In this example, a higher entropy of the score distribution over the set of dyskinesia classes 110 can reflect a higher uncertainty of the classification system 100 in the dyskinesia classification.

The classification system 100 can use tremor classifications 116 generated for the subject 102 in any of a variety of ways. A few example applications of tremor classifications 116 are described next.

In some implementations, the classification system 100 can generate a notification that indicates the tremor classification 116, and can provide the notification, e.g., to the subject 102 or to a healthcare provider of the subject 102. For instance, the classification system 100 can transmit the notification over a data communications network, e.g., as an email or a text message.

In some implementations, the classification system 100 can automatically store the tremor classification in an electronic medical record of the subject 102. For instance, the classification system 100 can interface with a database storing electronic medical records of the subject 102, e.g., by way of an application programming interface (API), and store the tremor classification 116 in an appropriate field in an electronic medical record of the subject 102.

In some implementations, the classification system 100 receives accelerometer data 104 (and optionally other inputs, such as video data or surface EMG data) for each time interval in a sequence of time intervals. The classification system 100 can process the accelerometer data 104 for each time interval to generate a corresponding tremor classification 116. The classification system 100 can thus generate a sequence of tremor classifications 116, where each tremor classification 116 corresponds to a respective time interval and characterizes the severity of the tremor of the subject 102 over the time interval. The sequence of tremor classifications can provide granular and real-time monitoring of the state and progression of tremors in the subject 102, without requiring the subject 102 to regularly see a physician or access a healthcare facility.

The classification system 100 can process the sequence of tremor classifications to classify a progression of Parkinson's disease in the subject into a progression state from a set of progression states. The set of progression states can include any appropriate number of progression states, e.g., three progression states, four progression states, or five progression states, and each progression state can correspond to a respective progression of Parkinson's disease in the subject. For instance, the set of progression states can include an “early stage” progression state, a “mid-stage” progression state, and a “late stage” progression state.

The classification system 100 can process the sequence of tremor classifications to classify the progression of Parkinson's in the subject 102 in any of a variety of ways. For instance, the classification system 100 can process the sequence of tremor classifications to determine, for each tremor class, the number of times over a preceding time window (e.g., a 1-week time window) that the tremor of the subject has been classified as being included in the tremor class. The classification system 100 can thus generate a frequency distribution over the set of tremor classes, where the frequency distribution associates each tremor class with the number of times the tremor of the subject has been classified as being included in the tremor class over the preceding time window. The classification system 100 can then classify the progression state of Parkinson's in the subject based on the frequency distribution over the set of tremor classes. For example, the classification system 100 can classify the progression of Parkinson's disease in the subject into a “late stage” progression state if the tremor of the subject has been classified into the tremor class associated with the highest severity of tremor at least a threshold number of times.

The classification system 100 can generate a recommendation for a treatment for the subject 102 based on the classification of the progression of Parkinson's disease in the subject 102. For instance, each progression state in the set of progression states can be associated with a corresponding recommended treatment, e.g., a dosage of a drug. After classifying the progression of Parkinson's disease in the subject 102 into a progression state (based on a sequence of tremor classifications), the classification system 100 can generate a recommendation to provide the subject 102 with the treatment associated with the progression state. The classification system 100 can provide the treatment recommendation to a user, e.g., a physician, by way of a user interface, e.g., a graphical user interface. A treatment can be applied to the subject 102 based at least in part on the treatment recommendation generated by the classification system 100. (The treatment can be, e.g., self-administered by the subject, or administered to the subject by a physician or another third-party).

The classification system 100 can allow objective and reproducible assessments of the effectiveness of treatments for Parkinson's, in particular, treatments that aim to reduce the severity of tremors in subjects. More specifically, the classification system 100 can automatically generate sequences of tremor classifications for subjects at any appropriate level of temporal granularity, e.g., by classifying the severity of the tremor in the subject every 10 seconds, or every minute, or every hour, etc. Sequences of tremor classifications generated for subjects receiving a treatment (e.g., a drug) provide an objective and reproducible metric for assessing the effect of the treatment, e.g., as opposed to relying on self-reporting by subjects, which may be inaccurate and inconsistent across subjects.

The classification system 100 can use dyskinesia classifications 118 generated for the subject 102 in any of a variety of ways. A few example applications of dyskinesia classifications 118 are described next.

In some implementations, the classification system 100 can generate a notification that indicates the dyskinesia classification 118, and can provide the notification, e.g., to the subject 102 or to a healthcare provider of the subject 102. For instance, the classification system 100 can transmit the notification over a data communications network, e.g., as an email or a text message.

In some implementations, the classification system 100 can automatically store the dyskinesia classification in an electronic medical record of the subject 102. For instance, the classification system 100 can interface with a database storing electronic medical records of the subject 102, e.g., by way of an application programming interface (API), and store the dyskinesia classification 118 in an appropriate field in an electronic medical record of the subject 102.

In some implementations, the classification system 100 receives accelerometer data 104 (and optionally other inputs, such as video data or surface EMG data) for each time interval in a sequence of time intervals. The classification system 100 can process the accelerometer data 104 for each time interval to generate a corresponding dyskinesia classification 118. The classification system 100 can thus generate a sequence of dyskinesia classifications 118, where each dyskinesia classification 118 corresponds to a respective time interval and characterizes the severity of the dyskinesia of the subject 102 over the time interval. The sequence of dyskinesia classifications can provide granular and real-time monitoring of the state and progression of dyskinesia in the subject 102, without requiring the subject 102 to regularly see a physician or access a hospital.

The classification system 100 can process the sequence of dyskinesia classifications to classify a progression of dyskinesia in the subject into a progression state from a set of progression states. The set of progression states can include any appropriate number of progression states, e.g., three progression states, four progression states, or five progression states, and each progression state can correspond to a respective progression of dyskinesia disease in the subject. For instance, the set of progression states can include an “early stage” progression state, a “mid-stage” progression state, and a “late stage” progression state.

The classification system 100 can process the sequence of dyskinesia classifications to classify the progression of dyskinesia in the subject 102 in any of a variety of ways. For instance, the classification system 100 can process the sequence of dyskinesia classifications to determine, for each dyskinesia class, the number of times over a preceding time window (e.g., a 1-week time window) that the dyskinesia of the subject has been classified as being included in the dyskinesia class. The classification system 100 can thus generate a frequency distribution over the set of dyskinesia classes, where the frequency distribution associates each dyskinesia class with the number of times the dyskinesia of the subject has been classified as being included in the dyskinesia class over the preceding time window. The classification system 100 can then classify the progression state of dyskinesia in the subject based on the frequency distribution over the set of dyskinesia classes. For example, the classification system 100 can classify the progression of dyskinesia disease in the subject into a “late stage” progression state if the dyskinesia of the subject has been classified into the dyskinesia class associated with the highest severity of dyskinesia at least a threshold number of times.

The classification system 100 can generate a recommendation for a treatment for the subject 102 based on the classification of the progression of dyskinesia in the subject 102. For instance, each progression state in the set of progression states can be associated with a corresponding recommended treatment, e.g., a dosage of a drug. After classifying the progression of dyskinesia in the subject 102 into a progression state (based on a sequence of dyskinesia classifications), the classification system 100 can generate a recommendation to provide the subject 102 with the treatment associated with the progression state. The classification system 100 can provide the treatment recommendation to a user, e.g., a physician, by way of a user interface, e.g., a graphical user interface. A treatment can be applied to the subject 102 based at least in part on the treatment recommendation generated by the classification system 100. (The treatment can be, e.g., self-administered by the subject, or administered to the subject by a physician or another third-party).

The classification system 100 can allow objective and reproducible assessments of the effectiveness of treatments for dyskinesia. More specifically, the classification system 100 can automatically generate sequences of dyskinesia classifications for subjects at any appropriate level of temporal granularity, e.g., by classifying the severity of dyskinesia in the subject every 10 seconds, or every minute, or every hour, etc. Sequences of dyskinesia classifications generated for subjects receiving a treatment (e.g., a drug) provide an objective and reproducible metric for assessing the effect of the treatment, e.g., as opposed to relying on self-reporting by subjects, which may be inaccurate and inconsistent across subjects.

FIG. 2 shows an example architecture of a classification neural network 200, e.g., that is included in the classification system 100 described with reference to FIG. 1. The classification neural network 200 is configured to receive a network input 106 that includes accelerometer data 104 characterizing movement of a subject 102 with Parkinson's disease. The network input 106 can include additional data characterizing the subject, e.g., video data or surface EMG data, as described above with reference to FIG. 1. The classification neural network 200 processes the network input 106 to generate one or both of: (i) a tremor classification 116 that characterizes a severity of a tremor of the subject 102, or (ii) a dyskinesia classification 118 that characterizes a severity of (and/or a presence of) dyskinesia in the subject 102.

The classification neural network 200 includes an encoder subnetwork 300, and one or both of: (i) a tremor subnetwork 204, and (ii) a dyskinesia subnetwork 206, which are each described in more detail next.

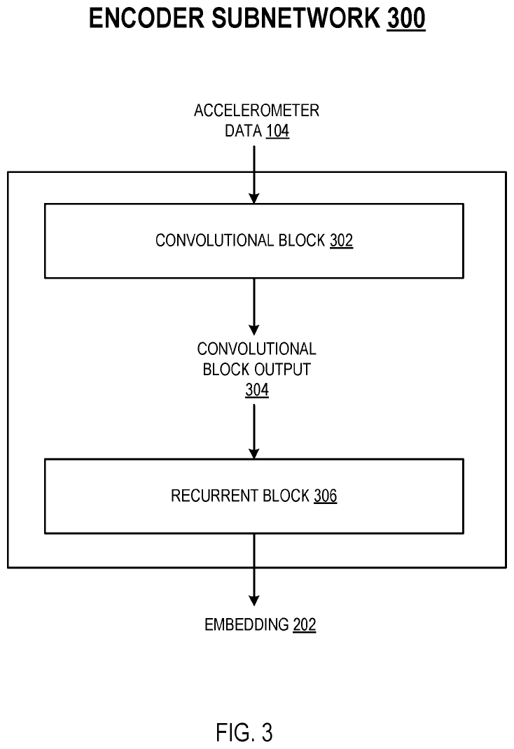

The encoder subnetwork 300 is configured to process the network input 106 to generate an embedding 202 of the network input 106 in a latent space. The encoder subnetwork 300 can have any appropriate neural network architecture that enables the encoder subnetwork 300 to generate an embedding 202 of a network input 106. In particular, the encoder subnetwork can include any appropriate types of neural network layers (e.g., convolutional layers, recurrent layers, attention layers, fully connected layers, etc.) in any number (e.g., 5 layers, 10 layers, or 50 layers) and in any appropriate configuration (e.g., as a linear sequence of layers). A specific example of an architecture of the encoder subnetwork 300 is described in more detail with reference to FIG. 3.

The network input 106 can include data from multiple modalities, e.g., accelerometer data, video data, surface EMG data, etc. The architecture of the encoder subnetwork 300 can be configured in any of a variety of possible ways to enable the encoder subnetwork 300 to process multi-modal data. For instance, the encoder subnetwork 300 can include a respective sequence of encoder neural network layers corresponding to each modality. For each modality, the encoder subnetwork 300 can process data derived from that modality using the corresponding sequence of encoder neural network layers to generate a modality-specific embedding of the data. The encoder subnetwork 300 can combine the modality-specific embedding of each modality included in the network input 106 to generate the embedding 202 of the network input 106. The encoder subnetwork 300 can combine the modality-specific embeddings, e.g., by concatenating the modality-specific embeddings, or by pooling (e.g., averaging, summing, or max pooling) the modality-specific embeddings.

The tremor subnetwork 204 is configured to process the embedding 202 of the network input 106 to generate a score distribution over the set of tremor classes 108. The tremor subnetwork 204 can have any appropriate neural network architecture that enables the tremor subnetwork 204 to generate a score distribution over a set of tremor classes. In particular, the tremor subnetwork can include any appropriate types of neural network layers (e.g., convolutional layers, recurrent layers, attention layers, fully connected layers, etc.) in any number (e.g., 5 layers, 10 layers, or 50 layers) and in any appropriate configuration (e.g., as a linear sequence of layers). In a specific example, the tremor subnetwork 204 can include a sequence of dense (fully connected) neural network layers.

The dyskinesia subnetwork 206 is configured to process the embedding 202 of the network input 106 to generate a score distribution over the set of dyskinesia classes 110. The dyskinesia subnetwork 206 can have any appropriate neural network architecture that enables the dyskinesia subnetwork 206 to generate a score distribution over a set of dyskinesia classes. In particular, the dyskinesia subnetwork can include any appropriate types of neural network layers (e.g., convolutional layers, recurrent layers, attention layers, fully connected layers, etc.) in any number (e.g., 5 layers, 10 layers, or 50 layers) and in any appropriate configuration (e.g., as a linear sequence of layers). In a specific example, the dyskinesia subnetwork 206 can include a sequence of dense (fully connected) neural network layers.

In some implementations, the classification neural network 200 includes the encoder subnetwork 300 and the tremor subnetwork 204, but does not include the dyskinesia subnetwork 206. In some implementations, the classification neural network 200 includes the encoder subnetwork 300 and the dyskinesia subnetwork 206, but does not include the tremor subnetwork 204. In some implementations, the classification neural network 200 includes both the tremor subnetwork 204 and the dyskinesia subnetwork 206.

FIG. 3 shows an example architecture of an encoder subnetwork 300 included in a classification neural network 200, e.g., as described with reference to FIG. 1, of a classification system 100, e.g., as described with reference to FIG. 1. In this example, the encoder subnetwork 300 includes: (i) a convolutional block 302 that includes one or more convolutional neural network layers, and (ii) a recurrent block 306 that includes one or more recurrent neural network layers.

The convolutional block 302 can be configured to process accelerometer data 104 characterizing the motion of a subject having Parkinson's disease, by one or more convolutional neural network layers, to generate a convolutional block output that has a lower temporal resolution than the accelerometer data. In particular, the accelerometer data can include a sequence of accelerometer data elements, e.g., where each accelerometer data element is represented as an 3D vector of vector of x-, y-, and z-accelerations. The convolutional block output can include a sequence of feature vectors, where the sequence of feature vectors has a shorter length than sequence of accelerometer data elements and thus has a lower temporal resolution than the sequence of accelerometer data elements.

The recurrent block 306 can be configured to process the convolutional block output 304, by one or more recurrent neural network layers, to generate an embedding 202 of the accelerometer data 104. The recurrent neural network layers can be, e.g., long short-term memory (LSTM) recurrent neural network layers, gated recurrent unit (GRU) recurrent neural network layers, or any other appropriate type of recurrent neural network layer. The recurrent neural network layers in the recurrent block 306 can be arranged in a sequence. Each recurrent neural network layer can be configured to sequentially process a sequence of feature vectors to generate, by a set of recurrent neural network layer operations, an updated sequence of feature vectors. The first recurrent neural network layer in the recurrent block 306 can receive the sequence of feature vectors included in the convolutional block output. Each subsequent recurrent neural network layer in the recurrent block 306 can receive the sequence of feature vectors generated by the preceding recurrent neural network layer in the recurrent block. The final recurrent neural network layer in the recurrent block 306 can generate a sequence of feature vectors that collectively define the embedding 202 of the accelerometer data 104.

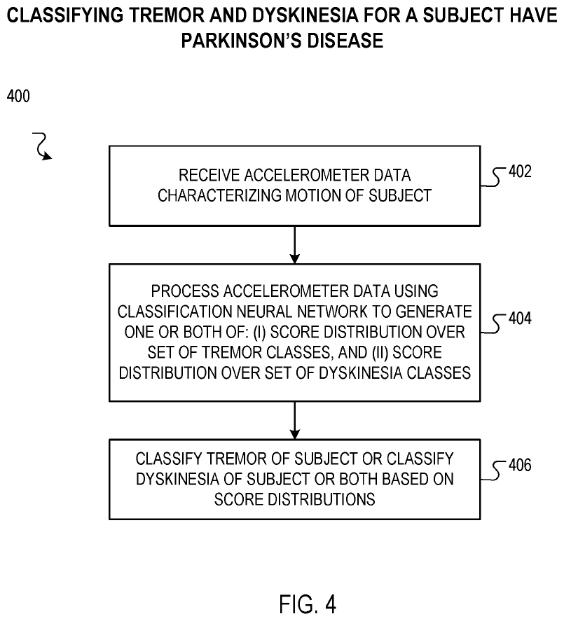

FIG. 4 is a flow diagram of an example process 400 for classifying a tremor of a subject having Parkinson's disease or for classifying dyskinesia of a subject having Parkinson's disease. For convenience, the process 400 will be described as being performed by a system of one or more computers located in one or more locations. For example, a classification system, e.g., the classification system 100 of FIG. 1, appropriately programmed in accordance with this specification, can perform the process 400.

The system receives accelerometer data characterizing motion of a subject having Parkinson's disease (402). The accelerometer data can be generated by a wearable device of the subject. The accelerometer data can be represented as one or more one-dimensional (1D) temporal signals, or as a two-dimensional (2D) spectrogram signal. Optionally, the system can receive additional data, e.g., video data characterizing the motion of the subject, or surface EMG data characterizing electrical potentials produced during muscle contractions of the subject.

The system processes a network input that includes the accelerometer data (and, optionally, additional data such as video data or surface EMG data) using a classification neural network, in accordance with values of a set of classification neural network parameters, to generate a score distribution over a set of tremor classes, or a score distribution over a set of dyskinesia classes, or both (404). The set of tremor classes includes multiple tremor classes, and each tremor class in the set of tremor classes corresponds to a respective severity of tremor. The set of dyskinesia classes includes multiple dyskinesia classes, and each dyskinesia class in the set of dyskinesia classes corresponds to a respective severity of dyskinesia.

The system classifies the tremor of the subject, the dyskinesia of the subject, or both (406). The system can classify the tremor of the subject based on the score distribution over the set of tremor classes, e.g., by classifying the tremor of the subject as being included in the tremor class with the highest score. The system can classify the dyskinesia of the subject based on the score distribution over the set of dyskinesia classes, e.g., by classifying the dyskinesia of the subject as being included in the dyskinesia class with the highest score.

FIG. 5 shows an example training system 500. The training system 500 is an example of a system implemented as computer programs on one or more computers in one or more locations in which the systems, components, and techniques described below are implemented.

The training system 500 is configured to train the classification neural network 200 included in the classification system 100 described with reference to FIG. 1. The classification neural network 200 is configured to process a network input that includes accelerometer data characterizing motion of a subject having Parkinson's disease to generate one or both of: (i) a score distribution over a set of tremor classes, and (ii) a score distribution over a set of dyskinesia classes, as described with reference to FIG. 1.

The classification neural network 200 can include an encoder subnetwork, and one or both of: (i) a tremor subnetwork, and (ii) a dyskinesia subnetwork, as described with reference to FIG. 2. The encoder subnetwork can process the network input, including the accelerometer data, to generate an embedding of the network input. The tremor subnetwork can process the embedding of the network input to generate a score distribution over the set of tremor classes. The dyskinesia subnetwork can process the embedding of the network input to generate a score distribution over the set of dyskinesia classes.

The training system 500 can train the classification neural network over a sequence of two stages. In the first stage, the training system 500 can pre-train the encoder subnetwork 300 of the classification neural network 200 to perform one or more auxiliary tasks. In the second stage, the training system 500 can train the classification neural network 200 to perform the tremor classification task, or the dyskinesia classification task, or both. Pre-training the encoder subnetwork 300 can provide an effective initialization of the parameter values of the encoder neural network, thus facilitating fine-tuning of the classification neural network to perform tremor classification or dyskinesia classification. In particular, the pre-training can encourage the encoder subnetwork 300 to generate embeddings that encode rich features characterizing the structure of accelerometer data (and, optionally, other types of data, e.g., video data or surface EMG data). Pre-training the encoder subnetwork 300 can enable the classification neural network 200 to achieve a higher prediction accuracy on the tremor classification task and the dyskinesia classification task while requiring less training data than would otherwise be necessary.

At the first stage of training, the training system 500 can initialize the set of parameters of the encoder subnetwork 300, e.g., to random values. The training system 500 can then train the encoder subnetwork 300 to perform an auxiliary task. A few examples of auxiliary tasks are described next.

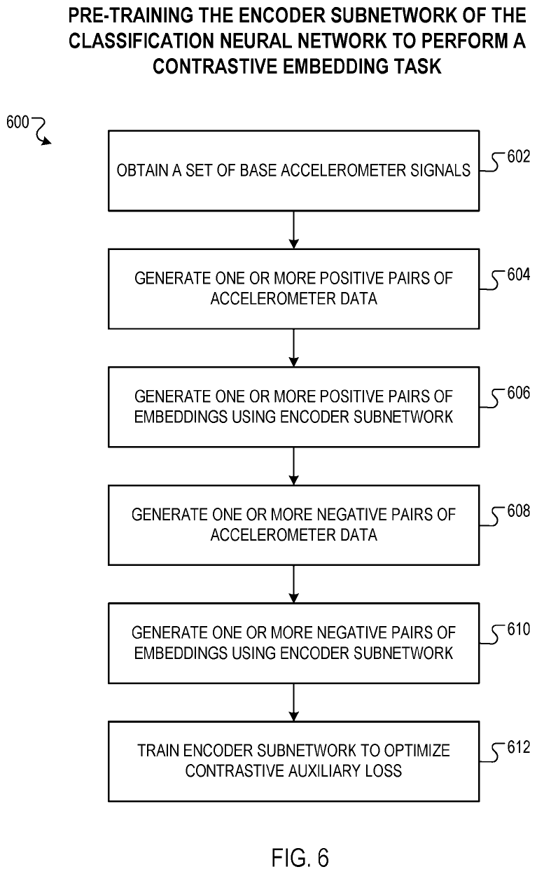

In some implementations, the training system 500 can pre-train the encoder subnetwork 300 to perform a contrastive embedding task 502. More specifically, the training system 500 can train the encoder subnetwork to generate similar embeddings for “positive” pairs of accelerometer signals, and dissimilar embeddings for “negative” pairs of accelerometer signals. A “pair” of accelerometer signals includes a first accelerometer signal and a second accelerometer signal. A “positive” pair of accelerometer signals can refer to a pair of accelerometer signals where the first accelerometer signal and the second accelerometer signal are both transformed version of a same underlying accelerometer signal. A “negative” pair of accelerometer signals can refer to a pair of accelerometer signals where the first accelerometer signal is derived from a different accelerometer signal than the second accelerometer signal. An example process for training the encoder subnetwork 300 to perform a contrastive embedding task 502 is described in more detail with reference to FIG. 6.

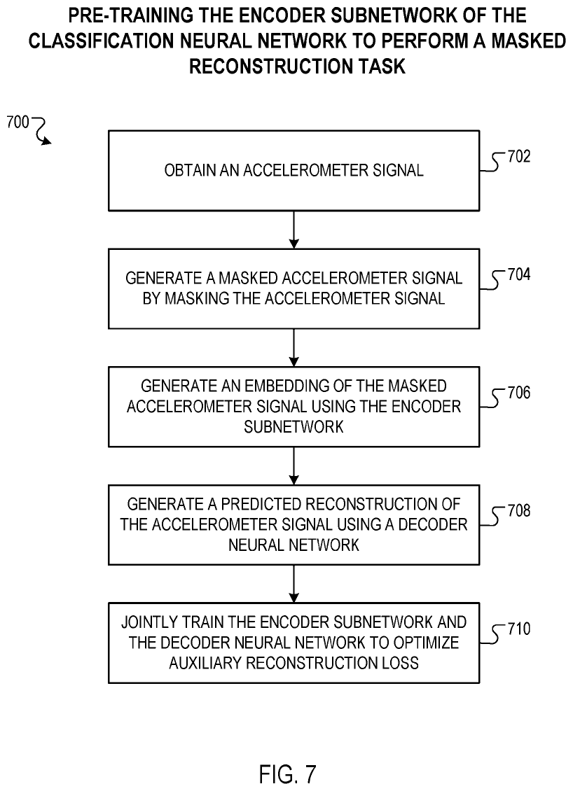

In some implementations, the training system 500 can pre-train the encoder subnetwork 300 to perform a masked reconstruction task 504. More specifically, the training system can train the encoder subnetwork to process a masked accelerometer signal to generate an embedding that enables accurate reconstruction of the full (unmasked) accelerometer signal. An example process for training the encoder subnetwork 300 to perform a masked reconstruction task 504 is described in more detail with reference to FIG. 7.

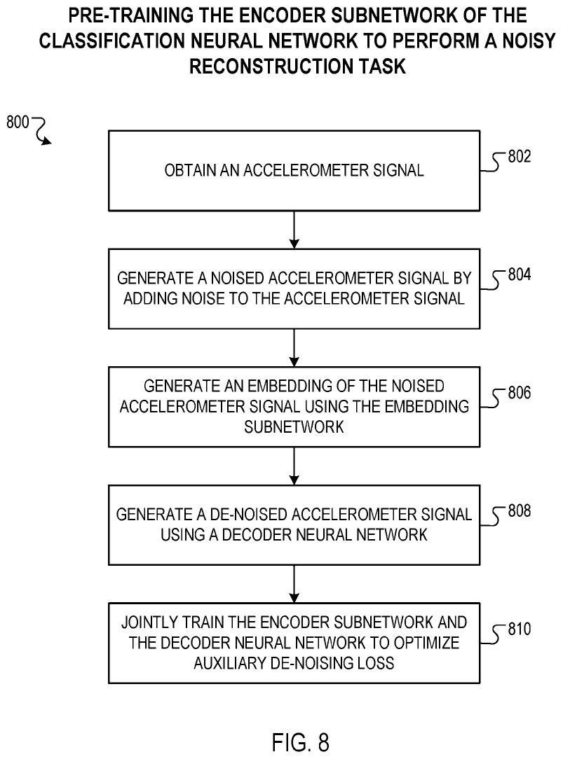

In some implementations, the training system 500 can pre-train the encoder subnetwork 300 to perform a noisy reconstruction task 506. More specifically, the training system can train the encoder subnetwork to process a noised accelerometer signal to generate an embedding that enables accurate reconstruction of the original (de-noised) accelerometer signal. An example process for training the encoder subnetwork 300 to perform a noisy reconstruction task 506 is described in more detail with reference to FIG. 8.

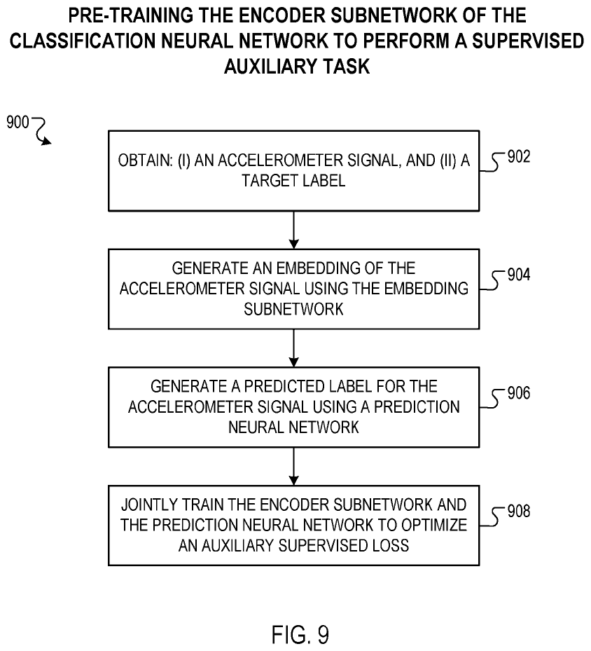

In some implementations, the training system 500 can pre-train the encoder subnetwork 300 to perform one or more supervised auxiliary tasks 508. More specifically, the training system can train the encoder subnetwork to process an accelerometer signal to generate an embedding that enables accurate prediction of one or more features of the accelerometer signal, e.g., a number of steps taken by a subject in a duration of time covered by the accelerometer signal, or an action performed by a subject in a duration of time covered by the accelerometer signal. An example process for training the encoder subnetwork to perform a supervised auxiliary task 508 is described in more detail with reference to FIG. 9.

Optionally, the training system 500 can pre-train the encoder subnetwork to perform multiple auxiliary tasks, i.e., rather than just a single auxiliary task. For instance, the training system 500 can pre-train the encoder subnetwork to perform two auxiliary tasks, or three auxiliary tasks, or four auxiliary tasks.

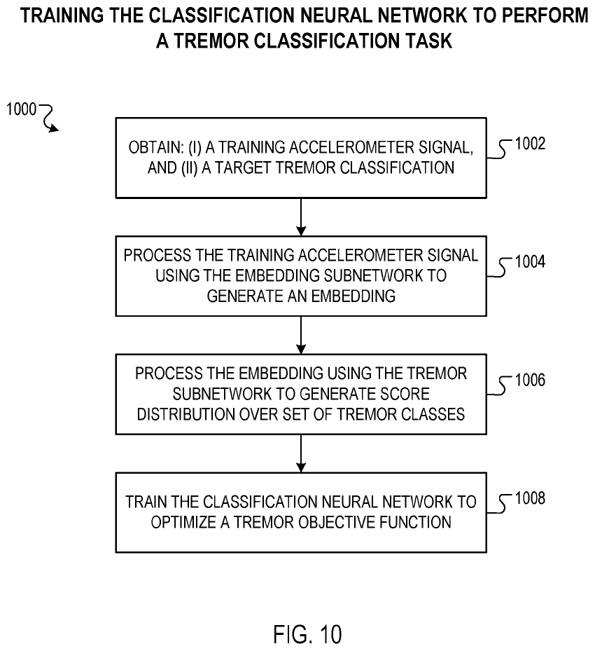

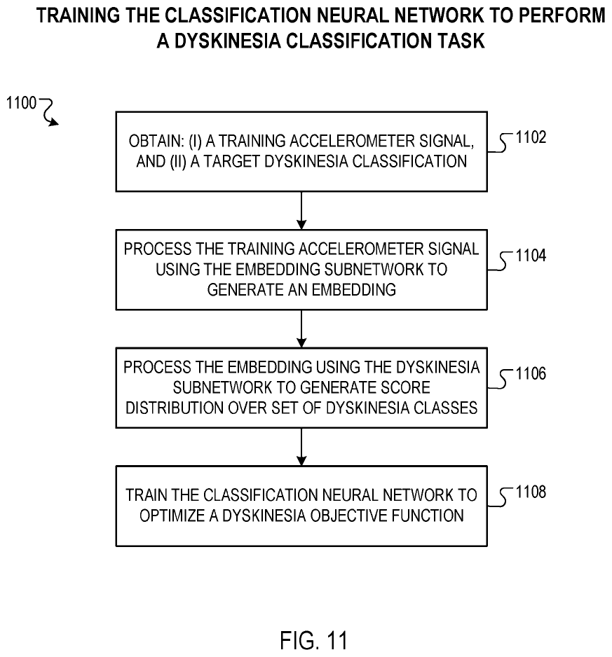

At the second stage of training, the training system 500 can initialize the set of parameters of the tremor subnetwork, the dyskinesia subnetwork, or both, e.g., to random values. (The set of parameters of the encoder subnetwork 300 can have the values determined during the pre-training of the encoder subnetwork at the first stage of training). The training system 500 can then train the classification neural network 200 to perform a tremor classification task 510, or a dyskinesia classification task 512, or both. An example process for training the classification neural network to perform a tremor classification task 510 is described in more detail with reference to FIG. 10. An example process for training the classification neural network to perform a dyskinesia classification task is described in more detail with reference to FIG. 11.

In some implementations, the training system 500 trains the classification neural network 200 to perform both a tremor classification task and a dyskinesia classification task. In these implementations, the training system 500 can exploit synergies that exist between the tremor classification task and the dyskinesia classification task in order to achieve a higher prediction accuracy on both tasks. More specifically, as part of training the classification neural network to perform the tremor classification task, the training system can backpropagate gradients of a tremor classification objective function through the tremor subnetwork and into the encoder subnetwork of the classification neural network. Similarly, as part of training the classification neural network to perform the dyskinesia classification task, the training system can backpropagate gradients of a dyskinesia classification objective function through the dyskinesia subnetwork and into the encoder subnetwork of the classification neural network. The parameter values of the encoder subnetwork can thus be jointly trained using training signals from both the tremor classification task and the dyskinesia classification task, thereby enabling the encoder subnetwork to learn to exploit synergies and commonalities between the tasks.

For convenience, the first stage of training-in particular, pre-training the encoder neural network—is described as being performed before the second stage of training—in particular, fine-tuning the classification neural network to perform tremor classification and/or dyskinesia classification. However, the two stages of training can overlap, e.g., such that the pre-training of the encoder neural network and the fine-tuning of the classification neural network are both performed at one or more training iterations.

After training the classification neural network 200, the training system can provide the classification neural network 200 for use by the classification system 100, as described with reference to FIG. 1.

FIG. 6 is a flow diagram of an example process 600 for pre-training the encoder subnetwork of the classification neural network to perform a contrastive embedding task. For convenience, the process 600 will be described as being performed by a system of one or more computers located in one or more locations. For example, a training system, e.g., the training system 500 of FIG. 5, appropriately programmed in accordance with this specification, can perform the process 600. The training system can iteratively perform the steps of the process 600 as part of pre-training the encoder subnetwork.

The system obtains a set of “base” accelerometer signals (602).

The system generates one or more positive pairs of accelerometer signals (604). To generate a positive pair of accelerometer signals, the system selects (e.g., randomly samples) a base accelerometer signal from the set of base accelerometer signals. The system generates a first transformed version of the base accelerometer signal, e.g., by randomly sampling a first transformation from a space of transformations, and applying the first transformation to the base accelerometer signal. The system generates a second transformed version of the base accelerometer signal, e.g., by randomly sampling a second transformation from the space of transformations, and applying the second transformation to the base accelerometer signal. The first transformed version and the second transformed version of the base accelerometer signal jointly define the positive pair of accelerometer signals.

The space of transformations can include, e.g., a flipping transformation (that includes flipping the accelerometer signals around the time axis), a reversing transformation (that includes reversing the direction of the time axis for the accelerometer signals), a zoom transformation (that includes cropping and resizing the accelerometer signals), a swapping transformation (that includes swapping the ordering of the accelerometer signals), a noising transformation (that includes adding random noise to the accelerometer signals), a resizing transformation (that includes modifying the amplitude of the accelerometer signals), and an identity transformation (that has no effect on accelerometer signals). In some cases, a positive pair of accelerometer signals can include an accelerometer signal that is identical to a base accelerometer signal, e.g., in situations where the system selects the identity transformation to apply to the base accelerometer signal.

The system generates one or more positive pairs of embeddings (606). In particular, for each positive pair of accelerometer signals, the system processes the first accelerometer signal using the encoder subnetwork to generate an embedding of the first accelerometer signal, and the system processes the second accelerometer signal to generate an embedding of the second accelerometer signal. The embedding of the first accelerometer signal and the embedding of the second accelerometer signal jointly define a positive pair of embeddings.

The system generates one or more negative pairs of accelerometer signals (608). To generate a negative pair of accelerometer signals, the system selects (e.g., randomly samples) a pair of base accelerometer signals, including a first base accelerometer signal and a second, different base accelerometer signal. The system generates a transformed version of the first base accelerometer signal, e.g., by randomly sampling a transformation from the space of transformations, and applying the transformation to the first base accelerometer signal. The system generates a transformed version of the second base accelerometer signal, e.g., by randomly sampling a transformation from the space of transformations, and applying the transformation to the second base accelerometer signal. The transformed versions of the first and second base accelerometer signals jointly define the negative pair of accelerometer signals. The system may select the identity transformation for either the first or second base accelerometer signal, such that the negative pair of accelerometer signals includes base accelerometer signals (rather than modified accelerometer signals).

The system generates one or more negative pairs of embeddings (610). In particular, for each negative pair of accelerometer signals, the system processes the first accelerometer signal using the encoder subnetwork to generate an embedding of the first accelerometer signal, and the system processes the second accelerometer signal to generate an embedding of the second accelerometer signal. The embedding of the first accelerometer signal and the embedding of the second accelerometer signal jointly define a negative pair of embeddings.

The system trains the encoder subnetwork to optimize an auxiliary contrastive loss that depends on the positive pairs of embeddings and the negative pairs of embeddings (612). More specifically, the auxiliary loss can, for each pair of embeddings, measure an error (e.g., a Euclidean distance) between the first embedding and the second embedding. In particular, for each positive pair of embeddings, the auxiliary loss can encourage a higher similarity between the first embedding and the second embedding. For each negative pair of embeddings, the auxiliary loss can encourage a lower similarity between the first embedding and the second embedding.

FIG. 7 is a flow diagram of an example process 700 for pre-training the encoder subnetwork of the classification neural network to perform a masked reconstruction task. For convenience, the process 700 will be described as being performed by a system of one or more computers located in one or more locations. For example, a training system, e.g., the training system 500 of FIG. 5, appropriately programmed in accordance with this specification, can perform the process 700. The training system can iteratively perform the steps of the process 700 as part of pre-training the encoder subnetwork.

The system obtains an accelerometer signal (702).

The system generates a masked accelerometer signal by masking a portion of the accelerometer signal (704). The system can mask a portion of the accelerometer signal by replacing a portion of the accelerometer signal with default data, e.g., predefined values (e.g., zero values) or random noise (e.g., sampled from a Normal distribution). The system can randomly select the portion of the accelerometer signal to be masked.

The system processes the masked accelerometer signal using the encoder subnetwork to generate an embedding of the masked accelerometer signal (706).

The system processes the embedding of the masked accelerometer signal using a decoder neural network to generate a predicted reconstruction of the accelerometer signal (708). The decoder neural network can have any appropriate neural network architecture that enables the decoder neural network to generate a predicted reconstruction of an accelerometer signal. In particular, the decoder neural network can include any appropriate types of neural network layers (e.g., convolutional layers, recurrent layers, attention layers, fully connected layers, etc.) in any number (e.g., 5 layers, 10 layers, or 50 layers) and in any appropriate configuration (e.g., as a linear sequence of layers).

The system jointly trains the encoder subnetwork and the decoder neural network to optimize an auxiliary reconstruction loss that measures an error in the predicted reconstruction of the accelerometer signal (710). The error can be measured, e.g., as an L₁ error, or as an L₂ error, or using any other appropriate error metric.

FIG. 8 is a flow diagram of an example process 800 for pre-training the encoder subnetwork of the classification neural network to perform a noisy reconstruction task. For convenience, the process 800 will be described as being performed by a system of one or more computers located in one or more locations. For example, a training system, e.g., the training system 500 of FIG. 5, appropriately programmed in accordance with this specification, can perform the process 800. The training system can iteratively perform the steps of the process 800 as part of pre-training the encoder subnetwork.

The system obtains an accelerometer signal (802).

The system generates a noised accelerometer signal by adding noise the accelerometer signal (804). The system can sample the noise from a predefined probability distribution, e.g., a Normal distribution.

The system processes the noised accelerometer signal using the encoder subnetwork to generate an embedding of the noised accelerometer signal (806).

The system processes the embedding of the noised accelerometer signal using a decoder neural network to generate a predicted de-noised accelerometer signal, i.e., a predicted reconstruction of the original (de-noised) accelerometer signal (808). The decoder neural network can have any appropriate neural network architecture that enables the decoder neural network to generate a predicted de-noised accelerometer signal. In particular, the decoder neural network can include any appropriate types of neural network layers (e.g., convolutional layers, recurrent layers, attention layers, fully connected layers, etc.) in any number (e.g., 5 layers, 10 layers, or 50 layers) and in any appropriate configuration (e.g., as a linear sequence of layers).

The system jointly trains the encoder subnetwork and the decoder neural network to optimize an auxiliary de-noising loss that measures an error in the de-noised accelerometer signal (810). More specifically, the auxiliary de-noising loss can measure an error between: (i) the original accelerometer signal, and (ii) the predicted de-noised accelerometer signal. The error can be measured, e.g., as an L₁ error, or as an L₂ error, or using any other appropriate error metric.

FIG. 9 is a flow diagram of an example process 900 for pre-training the encoder subnetwork of the classification neural network to perform a supervised auxiliary task. For convenience, the process 900 will be described as being performed by a system of one or more computers located in one or more locations. For example, a training system, e.g., the training system 500 of FIG. 5, appropriately programmed in accordance with this specification, can perform the process 900. The training system can iteratively perform the steps of the process 900 as part of pre-training the encoder subnetwork.

The system obtains: (i) an accelerometer signal, and (ii) a target label for the accelerometer signal (902). In some implementations, the target label for the accelerometer signal defines a number of steps taken by the subject in a duration of time covered by the accelerometer signal. In some implementations, the target label for the accelerometer signal defines an action performed by the subject in a duration of time covered by the accelerometer signal.

The system processes the accelerometer signal using the encoder subnetwork to generate an embedding of the accelerometer signal (904).

The system processes the embedding of the accelerometer signal using a prediction neural network to generate a prediction output that characterizes a predicted label for the accelerometer signal (906). In some implementations, the prediction output directly defines the predicted label. In some implementations, the prediction output defines a score distribution over a set of possible labels.

The system jointly trains the encoder subnetwork and the prediction neural network to optimize an auxiliary supervised loss that measures an error between: (i) the target label, and (ii) the prediction output characterizing the predicted label (908). The auxiliary supervised loss can measure the error, e.g., as a cross-entropy error, or as a squared error.

FIG. 10 is a flow diagram of an example process 1000 for training the classification neural network to perform a tremor classification task. For convenience, the process 1000 will be described as being performed by a system of one or more computers located in one or more locations. For example, a training system, e.g., the training system 500 of FIG. 5, appropriately programmed in accordance with this specification, can perform the process 1000. The training system can iteratively perform the steps of the process 1000 as part of training the classification neural network.

The system obtains: (i) a training accelerometer signal, and (ii) a target tremor classification for the training accelerometer signal (1002).

The system processes the training accelerometer signal using the embedding subnetwork to generate an embedding of the training accelerometer signal (1004).

The system processes the embedding of the training accelerometer signal using the tremor subnetwork to generate a score distribution over the set of tremor classes (1006).

The system trains the tremor subnetwork of the classification neural network to optimize a tremor objective function that measures an error between: (i) the score distribution over the set of tremor classes, and (ii) the target tremor classification (1008). The tremor objective function can measure the error, e.g., as a cross-entropy error.

The system determines gradients of the tremor objective function, e.g., using backpropagation, and then adjusts the values of the set of parameters of the tremor subnetwork using the gradients by an update rule of an appropriate gradient descent optimization technique, e.g., RMSprop or Adam. That is, the system backpropagates gradients of the tremor objective function through the tremor subnetwork. In some implementations, the system jointly trains the tremor subnetwork and the encoder subnetwork by backpropagating gradients through the tremor subnetwork and into the encoder subnetwork. In other implementations, the system freezes the parameter values of the encoder subnetwork after pre-training the encoder subnetwork to perform one or more auxiliary tasks, and does not adjust the parameter values of the encoder subnetwork to optimize the tremor objective function.

FIG. 11 is a flow diagram of an example process 1100 for training the classification neural network to perform a dyskinesia classification task. For convenience, the process 1100 will be described as being performed by a system of one or more computers located in one or more locations. For example, a training system, e.g., the training system 500 of FIG. 5, appropriately programmed in accordance with this specification, can perform the process 1100. The training system can iteratively perform the steps of the process 1100 as part of training the classification neural network.

The system obtains: (i) a training accelerometer signal, and (ii) a target dyskinesia classification for the training accelerometer signal (1102).

The system processes the training accelerometer signal using the embedding subnetwork to generate an embedding of the training accelerometer signal (1104).

The system processes the embedding of the training accelerometer signal using the dyskinesia subnetwork to generate a score distribution over the set of dyskinesia classes (1106).

The system trains the dyskinesia subnetwork of the classification neural network to optimize a tremor objective function that measures an error between: (i) the score distribution over the set of dyskinesia classes, and (ii) the target dyskinesia classification (1108). The dyskinesia objective function can measure the error, e.g., as a cross-entropy error.

The system determines gradients of the dyskinesia objective function, e.g., using backpropagation, and then adjusts the values of the set of parameters of the dyskinesia subnetwork using the gradients by an update rule of an appropriate gradient descent optimization technique, e.g., RMSprop or Adam. That is, the system backpropagates gradients of the dyskinesia objective function through the dyskinesia subnetwork. In some implementations, the system jointly trains the dyskinesia subnetwork and the encoder subnetwork by backpropagating gradients through the dyskinesia subnetwork and into the encoder subnetwork. In other implementations, the system freezes the parameter values of the encoder subnetwork after pre-training the encoder subnetwork to perform one or more auxiliary tasks, and does not adjust the parameter values of the encoder subnetwork to optimize the dyskinesia objective function.

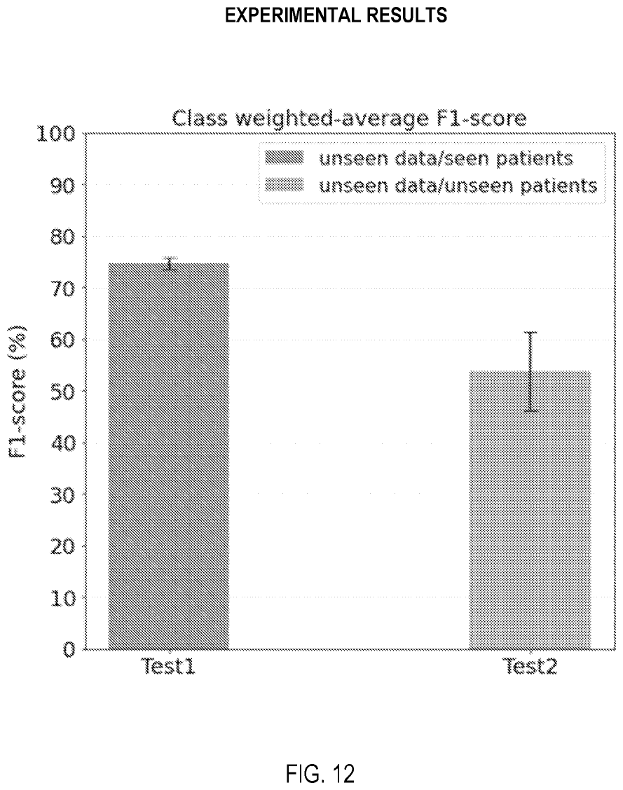

FIG. 12 illustrates an example of experimental results of the classification system in classifying tremors in subjects with Parkinson's disease.

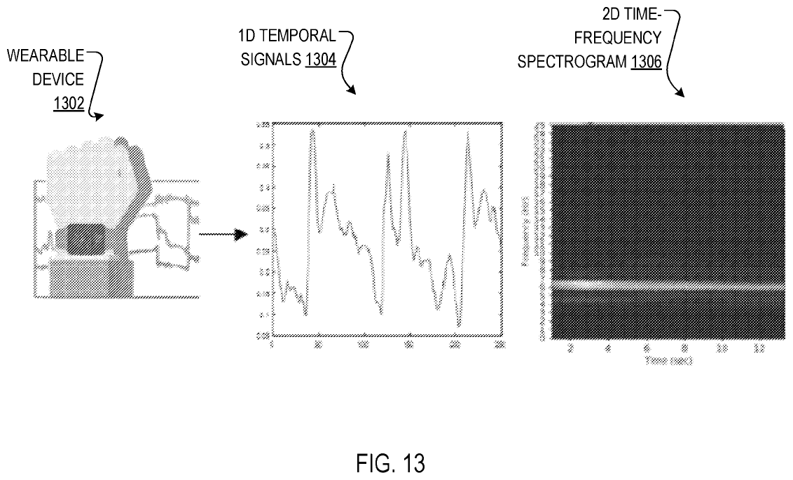

FIG. 13 illustrates: (i) a wearable device 1302, e.g., worn by a subject with Parkinson's disease; (ii) accelerometer data, generated by an accelerometer in the wearable device, represented as one or more 1D temporal signals 1304; and (iii) accelerometer data, generated by an accelerometer in the wearable device, represented as a 2D time-frequency spectrogram 1306.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers, the method comprising:
receiving accelerometer data characterizing motion of a subject having Parkinson's disease;
processing the accelerometer data using a tremor classification neural network, in accordance with values of a set of tremor classification neural network parameters, to generate a score distribution over a set of tremor classes,
wherein the set of tremor classes comprises multiple tremor classes,
wherein each tremor class in the set of tremor classes corresponds to a respective severity of tremor; and
classifying a tremor of the subject having Parkinson's disease based on the score distribution over the set of tremor classes.

2. The method of claim 1, wherein the accelerometer data is generated by a wearable device of the subject.

3. (canceled)

4. (canceled)

5. (canceled)

6. (canceled)

7. (canceled)

8. The method of claim 1, further comprising receiving video data characterizing motion of the subject;
wherein processing the accelerometer data using the tremor classification neural network comprises:
jointly processing the accelerometer data and the video data using the tremor classification neural network.

9. The method of claim 1, further comprising receiving surface electromyography (EMG) data characterizing electrical potentials produced during muscle contractions in the subject;
wherein processing the accelerometer data using the tremor classification neural network comprises:
jointly processing the accelerometer data and the surface EMG data using the tremor classification neural network.

10. The method of claim 1, wherein processing the accelerometer data using the tremor classification neural network to generate the score distribution over the set of tremor classes comprises:
processing the accelerometer data using a convolutional block, comprising one or more convolutional neural network layers, to generate a convolutional block output;
processing the convolutional block output using a recurrent block, comprising one or more recurrent neural network layers, to generate a recurrent block output; and
processing the recurrent block output using a dense block, comprising one or more dense neural network layers, to generate the score distribution over the set of tremor classes.

11. (canceled)

12. (canceled)

13. (canceled)

14. The method of claim 1, further comprising:
repeatedly performing the tremor classification over a sequence of time intervals to generate a sequence of tremor classifications, wherein each tremor classification in the sequence of tremor classifications corresponds to a respective time interval and classifies a tremor of the subject during the time interval.

15. The method of claim 14, further comprising:
processing the sequence of tremor classifications to classify a progression of Parkinson's disease in the subject into a progression state from a set of progression states.

16. (canceled)

17. (canceled)

18. The method of claim 1, wherein processing the accelerometer data using the tremor classification neural network to generate the score distribution over the set of tremor classes comprises:
processing the accelerometer data using an encoder subnetwork of the tremor classification neural network to generate an embedding of the accelerometer data in a latent space; and
processing the embedding of the accelerometer data in the latent space using a tremor subnetwork of the tremor classification neural network to generate the score distribution over the set of tremor classes.

19. The method of claim 18, further comprising:
processing the embedding of the accelerometer data in the latent space using a dyskinesia subnetwork of the tremor classification neural network to generate a score distribution over a set of dyskinesia classes,
wherein the set of dyskinesia classes comprises multiple dyskinesia classes,
wherein each dyskinesia class in the set of dyskinesia classes corresponds to a respective severity of dyskinesia.

20. The method of claim 19, further comprising:
classifying dyskinesia of the subject having Parkinson's disease based on the score distribution over the set of dyskinesia classes.

21. The method of claim 18, wherein the tremor classification neural network has been trained by operations comprising:
pre-training the encoder subnetwork of the tremor classification neural network to perform an auxiliary task, wherein the auxiliary task is not a tremor classification task; and
after pre-training the encoder subnetwork, training the tremor classification neural network to perform the tremor classification task.

22. The method of claim 21, wherein pre-training the encoder subnetwork of the tremor classification neural network to perform the auxiliary task comprises:
obtaining a base accelerometer signal;
generating a positive pair of embeddings in the latent space, comprising:
processing a first transformed version of the base accelerometer signal using the encoder subnetwork to generate a corresponding embedding in the latent space;
processing a second transformed version of the base accelerometer signal using the encoder subnetwork to generate a corresponding embedding in the latent space;
training the encoder subnetwork to optimize an auxiliary loss that depends on: (i) the embedding of the first transformed version of the base accelerometer signal, and (ii) the embedding of the second transformed version of the base accelerometer signal.

23. The method of claim 22, further comprising generating the first transformed version of the base accelerometer signal, comprising:
randomly sampling a first transformation from a space of transformations; and
applying the first transformation to the base accelerometer signal to generate the first transformed version of the base accelerometer signal.

24. The method of claim 22, further comprising generating the second transformed version of the base accelerometer signal, comprising:
randomly sampling a second transformation from a space of transformations; and
applying the second transformation to the base accelerometer signal to generate the second transformed version of the base accelerometer signal.

25. The method claim 22, wherein the auxiliary loss measures an error between: (i) embedding of the first transformed version of the base accelerometer signal, and (ii) the embedding of the second transformed version of the base accelerometer signal.

26. The method of claim 21, wherein pre-training the encoder subnetwork of the tremor classification neural network to perform the auxiliary task comprises:
obtaining an accelerometer signal;
generating a masked accelerometer signal by masking a portion of the accelerometer signal;
processing the masked accelerometer signal using the encoder subnetwork to generate an embedding of the masked accelerometer signal;
processing the embedding of the masked accelerometer signal using a decoder neural network to generate a predicted reconstruction of the accelerometer signal; and
jointly training the encoder subnetwork and the decoder neural network to optimize an auxiliary loss that measures an error in the predicted reconstruction of the accelerometer signal.

27. The method of claim 21, wherein pre-training the encoder subnetwork of the tremor classification neural network to perform the auxiliary task comprises:
obtaining an accelerometer signal;
generating a noised accelerometer signal by adding noise the accelerometer signal;
processing the noised accelerometer signal using the encoder subnetwork to generate an embedding of the noised accelerometer signal;
processing the embedding of the noised accelerometer signal using a decoder neural network to generate a de-noised accelerometer signal;
jointly training the encoder subnetwork and the decoder neural network to optimize an auxiliary loss that measures an error in the de-noised accelerometer signal.

28. (canceled)

29. (canceled)

30. (canceled)

31. The method of claim 1, wherein training the tremor classification neural network to perform the tremor classification task comprises:
obtaining: (i) a training accelerometer signal, and (ii) a target tremor classification for the training accelerometer signal;
processing the training accelerometer signal using the embedding subnetwork to generate an embedding of the training accelerometer signal;
processing the embedding of the training accelerometer signal using the tremor subnetwork to generate a score distribution over the set of tremor classes; and
training the tremor subnetwork of the tremor classification neural network to optimize a tremor objective function that measures an error between: (i) the score distribution over the set of tremor classes, and (ii) the target tremor classification.

32. (canceled)

33. A system comprising:
one or more computers; and
one or more storage devices communicatively coupled to the one or more computers, wherein the one or more storage devices store instructions that, when executed by the one or more computers, cause the one or more computers to perform operations comprising:
receiving accelerometer data characterizing motion of a subject having Parkinson's disease;
processing the accelerometer data using a tremor classification neural network, in accordance with values of a set of tremor classification neural network parameters, to generate a score distribution over a set of tremor classes.
wherein the set of tremor classes comprises multiple tremor classes,
wherein each tremor class in the set of tremor classes corresponds to a respective severity of tremor; and
classifying a tremor of the subject having Parkinson's disease based on the score distribution over the set of tremor classes.

34. One or more non-transitory computer storage media storing instructions that when executed by one or more computers cause the one or more computers to perform operations comprising:
receiving accelerometer data characterizing motion of a subject having Parkinson's disease;
processing the accelerometer data using a tremor classification neural network, in accordance with values of a set of tremor classification neural network parameters, to generate a score distribution over a set of tremor classes,
wherein the set of tremor classes comprises multiple tremor classes,
wherein each tremor class in the set of tremor classes corresponds to a respective severity of tremor; and
classifying a tremor of the subject having Parkinson's disease based on the score distribution over the set of tremor classes.
